(19) United States
(12) Patent Application Publication
(10) Pub. No.: US 2015/0202415 A1
(43) Pub. Date: Jul. 23, 2015

(54) INSERTION DEVICE

(71) Applicant: BIOTRONIK AG, Buelach (CH)

(72) Inventor: Amir Fargahi, Buelach (CH)

(21) Appl. No.: 14/565,642

(22) Filed: Dec. 10, 2014

Related U.S. Application Data
(60) Provisional application No. 61/929,993, filed on Jan. 22, 2014.

(51) Int. Cl. A61M 25/10

(52) U.S. Cl. CPC A61M 25/104; A61M 2025/1072

(57) ABSTRACT

An insertion device (100), in particular a guide catheter (110) and/or balloon catheter (120), for insertion into an animal and/or human body, wherein at least one catheter segment (10, 20) is provided, in which a hollow space (113, 32) and a fluid chamber (115, 125) are arranged around a central region (34), wherein the hollow space (113, 32) and the fluid chamber (115, 125) are separated by a wall (50) and the wall (50) has at least one wall opening (52a, 52b), which is sealed by a resilient membrane (40a, 40b), and wherein the resilient membrane (40a, 40b) is designed, with a sufficient pressure difference between the fluid chamber (115, 125) and the hollow space (113, 32), to bear against a bearing region (111, 30) in the hollow space (113, 32), said bearing region being arranged opposite the at least one wall opening (52a, 52b).

### CROSS REFERENCE TO RELATED APPLICATIONS

This invention claims benefit of priority to U.S. provisional patent application Ser. No. 61/929,993 filed on Jan. 22, 2014, the content of which is herein incorporated by reference in its entirety.

### TECHNICAL FIELD

The invention relates to an insertion device, in particular a balloon catheter or a guide catheter, for insertion into an animal and/or human body.

### BACKGROUND

It is known to use what are known as balloon catheters for percutaneous transluminal coronary angioplasty (PTCA) or for percutaneous transluminal angioplasty (PTA) in order to widen a narrowed or closed blood vessel so as to enable an interrupted flow of blood in the vessel. To this end, a guide catheter is normally inserted via a blood vessel and the balloon catheter is advanced thereby to the location to be treated. A balloon is located at the distal end of the balloon catheter and is expanded in the vascular constriction by being acted on by pressure by means of a fluid. Further similar indications for the use of balloon catheters include what is known as valvuloplasty and what is known as renal sympathetic denervation.

The balloon is connected to catheters before insertion into the body and has to be secured such that it can be positioned accurately and without complication at the site of use. A frequent problem here is that the balloon can shift, which may lead to a wide range of complications when the balloon, acted on by pressure, is expanded.

A balloon catheter comprising a guide wire and three balloons arranged in series is known from US 2010/0262076 A1. A control balloon is used for positioning before the vascular constriction or the vascular closure. A stabilizing balloon is used to fix the balloon catheter in the guide catheter. An anchor balloon arranged between the control balloon and stabilizing balloon is used to provide further anchoring in the bodily vessel.

### SUMMARY

The object of the invention is to specify an improved insertion device, with which the insertion device, in particular a balloon connected thereto, can be positioned in a highly precise manner.

The object is achieved in accordance with the invention by the features of claim 1. Favorable embodiments and advantages of the invention will emerge from the further claims, the drawings and the description.

An insertion device is provided, in particular a guide catheter and/or balloon catheter, for insertion into an animal and/or human body, wherein at least one catheter segment is provided, in which a hollow space and a fluid chamber are arranged around a central region, wherein the hollow space and the fluid chamber are separated by a wall and the wall has at least one wall opening, which is sealed by a resilient membrane. The resilient membrane is designed, with a sufficient pressure difference between the fluid chamber and hollow space, to come to bear against a bearing region in the hollow space, the bearing region being arranged opposite the at least one wall opening.

Here, depending on the embodiment, the membrane can reach through the at least one wall opening or can be pressed away from the wall opening.

When the membrane bears against the bearing region, the position and orientation of the catheter segment and bearing region relative to one another are fixed. If the pressure is reduced again, the membrane reverts back into its starting position, and the position and orientation are free again. Expressed in a simplified manner, the membrane therefore acts as a brake for fixing purposes. A fixing system, in particular for balloon catheters, can advantageously be created which is independent of the outer balloon surface. This can be charged with active ingredient and/or coated with a hydrophilic or hydrophobic coating in order to reduce friction. The at least one wall opening can be formed as a round opening or as a slot.

A sufficient pressure is expediently in the region of the normal nominal pressure in the event of balloon expansion and is at least 50% of the nominal pressure. Nominal pressure means the pressure that is applied to the balloon in the respective application. The resilience of the membrane can be designed accordingly. A favorable pressure range is at least 0.5 bar, preferably at least 1.0 bar, in particular 2-3 bar. A value of half the nominal pressure can also be used depending on the application.

In accordance with a favorable embodiment, the bearing region can be arranged around the same central region as the fluid chamber and hollow space or can be arranged in the central region. This gives a simple and reliable geometry, which does not interfere with the usual design of catheters.

In accordance with a favorable embodiment, the at least one wall opening can be formed by perforations. The pierced regions of the inner or outer tube retain sufficient stability, whereas a sufficient adhesive force of the membrane as it bears against the bearing region can be applied at the same time by a plurality of openings, through which the membrane is acted on by pressure.

In accordance with a favorable embodiment, the hollow space can surround the fluid chamber. This is favorable in a guide catheter. The membrane can be arranged for example on an outer face of an outer tube. In this case, the membrane can be pressed by a fluid from the inner fluid chamber, which applies a sufficient pressure, away from the at least one wall opening and outwardly into the hollow space and against an inner wall of a sleeve of a port or of a guide catheter depending on the indication.

A port is understood to mean a simple access (similar to a tube) into the respective bodily vessel. This access is sterile and is fixed to the patient in the bodily vessel in the proximal region. Ports of this type are used predominantly with peripheral accesses. The port does not reach very far into the bodily vessel.

A guide catheter is used in more complex applications. Here, the actual catheter is guided through the guide catheter to the respective position of the application. Accordingly, the guide catheter reaches into the bodily vessel practically as far as the site of application. The guide catheter is likewise fixed at the location of the external access into the patient in the proximal region.

The following applications for the fixing of the catheter with respect to the patient thus emerge for the brake according to the invention: The catheter is fixed with respect to the port or the catheter is fixed with respect to the guide catheter.

In accordance with a favorable embodiment, the fluid chamber may surround the hollow space. This is favorable in a balloon catheter. The membrane may be arranged for example at the edge of the guide wire lumen. In this case, the membrane can reach through the at least one wall opening in the hollow space and can be pressed against the guide wire of the insertion device by a fluid from the outer fluid chamber, which applies a sufficient pressure. Here, the bearing region may advantageously be arranged in a balloon catheter.

In accordance with a favorable embodiment, the balloon and/or the membrane can be formed from flexible material, such as nylon or polyamide.

In accordance with a further favorable embodiment, the balloon and/or the membrane can be formed from a material selected from the group consisting of ethylene vinyl acetate, polyvinyl chloride, olefin copolymers, olefin homopolymers, polyethylene, polyethylene types (such as PE-HD or HDPE (with weakly branched polymer chains, therefore high density polymer chains, wherein “HD” stands for “high density”), PE-LD or LDPE (with strongly branched polymer chains, therefore low density polymer chains, wherein “LD” stands for “low density”), PE-LLD or LLDPE (linear low density polyethylene, of which the polymer molecule has only short branchings, wherein “LLD” stands for “linear low density”), PE-HMW (high molecular weight polyethylene, wherein the polymer chains are longer than in PE-HD, PE-LD or PE-LLD, wherein “HMW” stands for “high molecular weight”), PE-UHMW (ultra high molecular weight polyethylene with a mean molar mass of up to 6000 kg/mol and a density of 0.93-0.94 g/cm³, wherein “UHMW” stands for “ultra high molecular weight”)), polyetheretherketone (PEEK, as a thermoplastic resistant to high temperature which belongs to the substance group of polyaryletherketones), polyurethane, silicone, polyethylene terephthalate (PET, a thermoplastic from the family of polyesters, produced by polycondensation), polyether block amide (PEBA, a thermoplastic elastomer, known under the trade name PEBAX from the manufacturer Arkema), acrylonitrile polymers, acrylonitrile copolymers, acrylonitrile mixtures, resins, in particular ionomer resins.

In accordance with a favorable embodiment, the guide wire, balloon catheter and guide catheter can be fixed relative to one another in their relative orientation when pressure is applied to the resilient membrane. In this state, there is no relative movement between the balloon of the balloon catheter, the fixed guide wire and the guide catheter. As the balloon is expanded, the membrane or the membranes presses/press the guide wire and the guide catheter or the port together and holds these in place securely. The position of the balloon is therefore fixed. Once the balloon is relaxed, the resilient membrane reverts back to its original state.

It is also conceivable to fix just some of the components, for example only the balloon with respect to the guide wire or the balloon with respect to the guide catheter. The fluid supply in the insertion device can be designed accordingly for this purpose.

The insertion device can be easily operated by a user and requires no additional training and can be produced for PTA and PTCA balloons with only low outlay.

### DESCRIPTION OF THE DRAWINGS

The invention is explained in greater detail hereinafter by way of example on the basis of exemplary embodiments illustrated in drawings, in which:

FIG. 1 shows a schematic view of an insertion device with guide catheter and balloon catheter;

FIG. 2 shows a schematic sectional view through a region of a balloon catheter in accordance with an exemplary embodiment of the invention with relaxed balloon;

FIG. 3 shows a schematic sectional view through a region of the balloon catheter according to FIG. 2 with balloon acted on by pressure and fixed on a guide wire;

FIG. 4 shows a sectional view through a region of a guide catheter in accordance with an exemplary embodiment of the invention with relaxed outer tube;

FIG. 5 shows a sectional view through a region of the guide catheter according to FIG. 4 with outer tube acted on by pressure and fixed to a wall;

FIG. 6 shows a cross-sectional view through the guide catheter, balloon catheter and guide wire with relaxed balloon; and

FIG. 7 shows a cross-sectional view through the guide catheter, balloon catheter and guide wire with balloon acted on by pressure.

### DETAILED DESCRIPTION

In the figures, functionally like elements or elements acting in an identical manner are denoted in each case by the same reference signs. The figures are schematic illustrations of the invention. They do not show specific parameters of the invention. Furthermore, the figures reproduce merely typical embodiments of the invention and are not intended to limit the invention to the illustrated embodiments.

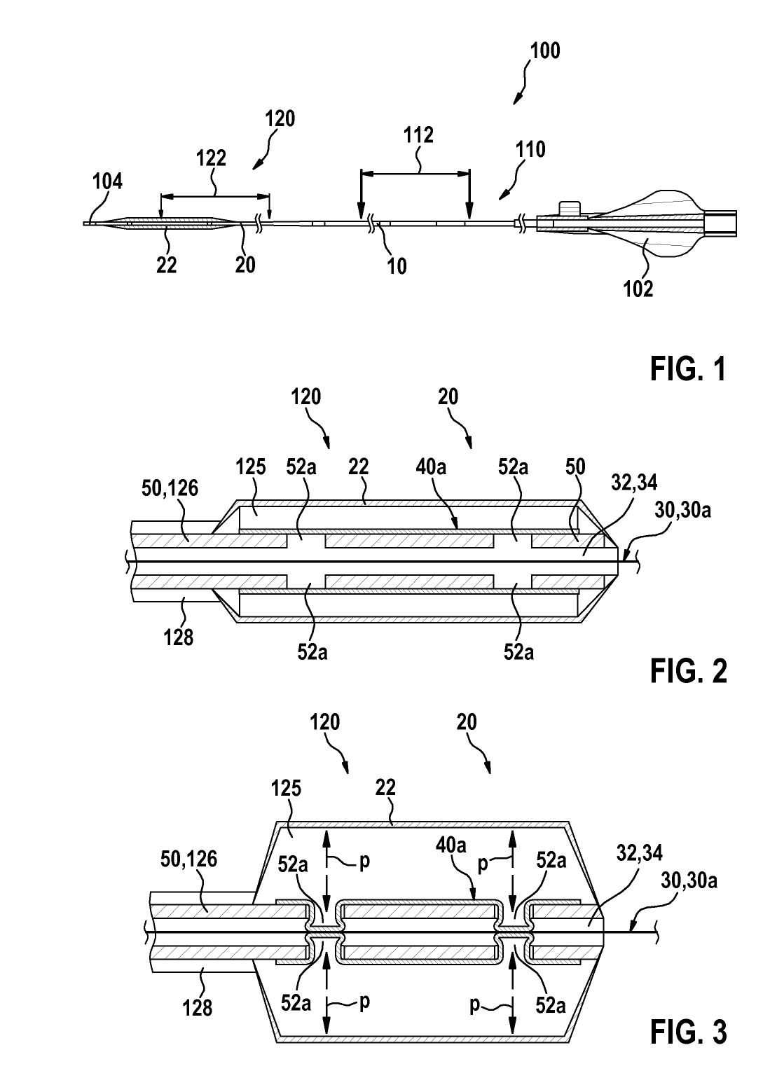

FIG. 1 shows a view of an insertion device 100 comprising a guide catheter 110 at its proximal end 102 and a balloon catheter 120 at its distal end 104. A guide wire (not shown) is guided through the balloon catheter 120. A balloon 20 is arranged at the distal end 104 and is to be expanded in the area of use so as to remedy a vascular constriction or a vascular closure.

The positions 112 and 122 denote favorable regions for providing a fixing of the guide catheter 110 in the region 112 and of the balloon catheter 120 in the region 122. A fixing may be provided in both regions 112, 122 or alternatively in just one of the regions 112, 122.

FIG. 2 shows a section through a catheter segment 20 of a balloon catheter 120 in accordance with an exemplary embodiment of the invention with relaxed balloon 22, not acted on by pressure. A guide wire 30a is guided through the balloon catheter 120 in a central region 34 in a hollow space 32. The balloon catheter 120 has at least one tube 126 (also referred to as a shaft), which is surrounded by the balloon 22 and of which the interior forms the central region 34. A fluid chamber 125 is arranged between the balloon 22 and the outer face of the tube 126 and surrounds the tube 126, it being possible for a fluid to be guided through the fluid chamber in the conventional manner so as to act on the balloon 22 with pressure when said balloon is to be expanded. The wall of the tube 126 forms a wall 50 between the hollow space 32 and fluid chamber 125. The balloon sleeve transitions at the proximal end into an outer tube 128, which surrounds the tube 126.

In the catheter segment 20, in which the balloon 22 is arranged, lateral openings 52a are provided in the wall 50, that is to say in the tube 126. The lateral openings 52a are covered by a resilient membrane 40a. In the shown example, the membrane 40a is slid over the tube 126 in the catheter segment 20 as a tube piece and surrounds the tube 126 in this region. The membrane 40a is located in the fluid chamber 125. An asymmetric arrangement is also conceivable, in which the membrane 40a is arranged only in strips or on one side on the periphery of the tube 126. The fluid chamber 125 and hollow space 32 surround the central region 34 and therefore also the guide wire 30a.

The balloon 20 and/or the membrane 40a can be formed by pliable materials, such as in particular ethylene vinyl acetate, polyvinyl chloride, olefin copolymers, olefin homopolymers, polyethylene types (such as PE-HD or HDPE, PE-LD or LDPE, PE-LLD or LLDPE, PE-HMW, PE-UHMW), polyetheretherketone, polyurethane, silicone, polyethylene terephthalate, polyether block amide (PEBA), acrylonitrile polymers, acrylonitrile copolymers, acrylonitrile mixtures, resins, in particular ionomer resins. Other suitable materials may also be used.

FIG. 3 shows a section through the catheter segment 20 of the exemplary embodiment in FIG. 2 with balloon 22 acted on by pressure and expanded. A high pressure p from a few bar to 20 bar is built up by the fluid supplied to the fluid chamber 125, and the balloon 22 is expanded accordingly. At the same time, the pressure p built up by the fluid presses the membrane 40a inwardly through the lateral openings 52a into the central region 34 until the membrane 40a contacts the guide wire 30a and fixes this in place.

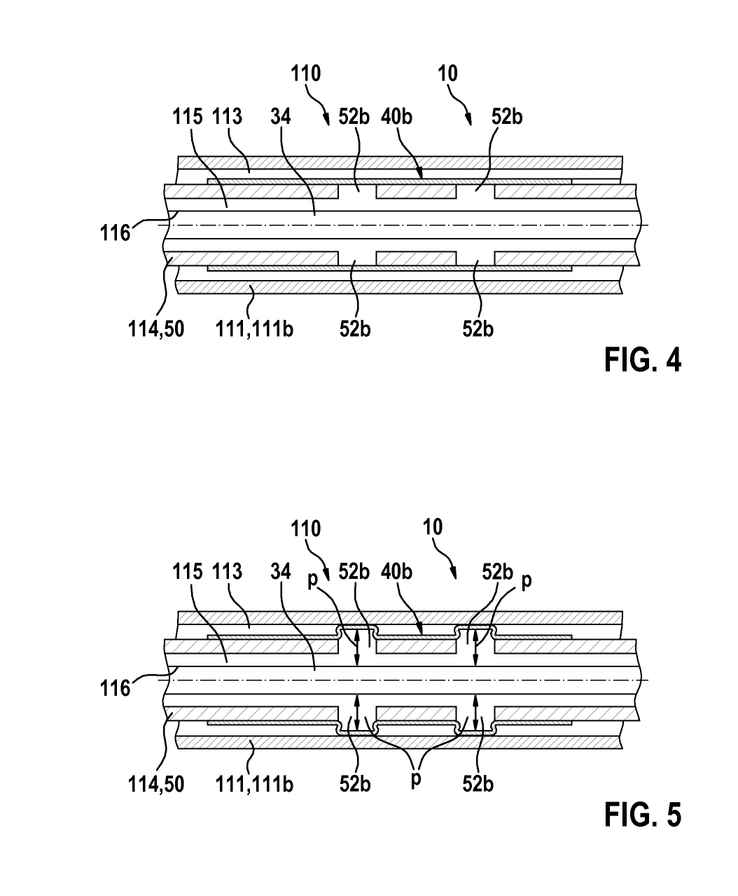

The guide wire 30a in this region forms a bearing region 30. The position and the orientation of balloon 22 and guide wire 30a are now fixed as long as the membrane 40a presses against the guide wire 30a. If the pressure p is reduced, the membrane 40a moves back again from its bearing region 30 into its starting position and releases the guide wire 30a. Due to the quasi “concentric” arrangement of balloon 22, fluid chamber 125, membrane 40a, tube 126, hollow chamber 32 and guide wire 30a, the function of the fixing of the balloon 22 with respect to the guide wire 30a can be easily implemented structurally. The expression “concentrically arranged” is to be understood to mean that either the hollow space surrounds the fluid chamber or the fluid chamber surrounds the hollow space. Due to the flexibility of the insertion device, the term “concentric” is, of course, not to be understood strictly geometrically. FIG. 4 shows a section through a segment 10 of a guide catheter 110 in accordance with an exemplary embodiment of the invention with outer tube 114 not acted on by pressure.

An inner tube 116 (also referred to as a shaft) is guided through the guide catheter 110 and is surrounded by an outer tube 114 (also referred to as a shaft). The interior of the inner tube 116 forms a central region 34 of the guide catheter 110. A fluid chamber 115 is formed between the inner tube 116 and outer tube 114 and surrounds the inner tube 116 and therefore the central region 34. A fluid can be guided through the fluid chamber 115 in order to apply pressure to the guide catheter 110. A sleeve 111b of a port is arranged around the outer tube 114 at a distance, whereby a hollow space 113 is defined. The outer tube 114 forms a wall 50 between the fluid chamber 115 and hollow space 113. The wall has wall openings 52b in the catheter segment 10.

A membrane 40b is arranged in the catheter segment and surrounds the outer tube 114 symmetrically and covers lateral openings 52b arranged there. An asymmetric arrangement of the membrane 40b is also conceivable, for example only at specific regions of the periphery of the outer tube 114.

The membrane 40a can be formed by pliant materials or by other materials, for example semi-solid materials, such as in particular ethylene vinyl acetate, polyvinyl chloride, olefin copolymers, olefin homopolymers, polyethylene types (such as PE-HD or HDPE, PE-LD or LDPE, PE-LLD or LLDPE, PE-HMW, PE-UHMW), polyetheretherketone, polyurethane, silicone, polyethylene terephthalate, polyether block amide (PEBA), acrylonitrile polymers, acrylonitrile copolymers, acrylonitrile mixtures, resins, in particular ionomer resins. Other suitable materials may also be used.

FIG. 5 shows a section through the catheter segment 10 of the exemplary embodiment in FIG. 4 with guide catheter 110 acted on by pressure. A fluid passes through the fluid chamber 115 into the catheter segment 10, where it builds up a pressure from a few bar to 20 bar depending on the intended use. The pressure p built up by the fluid presses the membrane 40b outwardly from the lateral openings 52b until the membrane 40b contacts the sleeve 111b, which forms a bearing region 111, and fixes this in place. The position and orientation of outer tube 114 and port are now fixed as long as the membrane 40b presses against the bearing region 111. If the pressure p is reduced, the membrane 40b moves back again from its bearing region 111 into its starting position and releases the guide catheter 110. Due to the quasi “concentric” arrangement of the sleeve 111b, hollow chamber 113, membrane 40b, outer tube 114, fluid chamber 115 and inner tube 116, the function of the fixing of the guide catheter 110 with respect to the port can be easily implemented structurally.

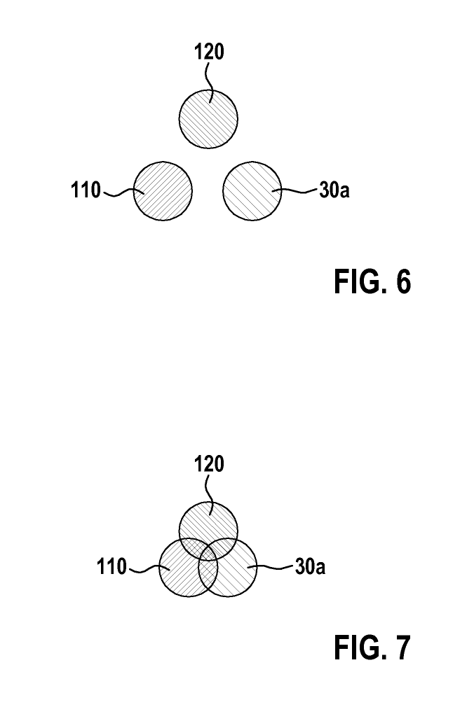

FIGS. 6 and 7 illustrate the action of the described fixing system of the insertion device. FIG. 6 shows symbolically a cross section through a guide catheter 110, a balloon catheter 120 and a guide wire 30a of an insertion device corresponding to FIG. 1 with a relaxed balloon (not illustrated). The positioning of the aforesaid components relative to one another is not fixed.

FIG. 7 shows symbolically a cross section through a guide catheter 110, a balloon catheter 120 and a guide wire 30a of an insertion device corresponding to FIG. 1 with a balloon acted on by pressure (not illustrated). There is no relative movement between the balloon and the fixed guide wire and/or guide catheter. Once the balloon has deflated, the resilient membranes revert back to their original state.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

### LIST OF REFERENCE SIGNS

100 insertion device

102 proximal end

104 distal end

110 guide catheter

111 sleeve/port/bearing region

112 fixing position of the guide catheter

113 hollow space

114 outer tube

115 fluid chamber

116 inner tube

120 balloon catheter

122 fixing position of the guide wire

125 fluid chamber

126 tube

128 sleeve

10 catheter segment

20 catheter segment

22 balloon

24 lumen

26 balloon catheter

30 bearing region

30a guide wire

32 hollow space

34 central region

40a, 40b membrane

50 wall

52a, 52b opening

p pressure

What is claimed is:

1. An insertion device, in particular a guide catheter and/or balloon catheter, for insertion into an animal and/or human body, wherein at least one catheter segment is provided, in which a hollow space and a fluid chamber are arranged around a central region, wherein the hollow space and the fluid chamber are separated by a wall and the wall has at least one wall opening, which is sealed by a resilient membrane, and wherein the resilient membrane is designed, with a sufficient pressure difference between the fluid chamber and the hollow space, to bear against a bearing region in the hollow space, the bearing region being arranged opposite the at least one wall opening.

2. The insertion device of claim 1, wherein the bearing region is arranged around the same central region as the fluid chamber and the hollow space or is arranged in the central region.

3. The insertion device of claim 1, wherein the at least one wall opening is formed by perforations.

4. The insertion device of claim 1, wherein the hollow space surrounds the fluid chamber.

5. The insertion device of claim 4, wherein the bearing region is associated with an inner wall of a sleeve of the insertion device.

6. The insertion device of claim 1, wherein the bearing region is arranged in a guide catheter.

7. The insertion device of claim 1, wherein the fluid chamber surrounds the hollow space.

8. The insertion device of claim 7, wherein the bearing region is associated with a guide wire.

9. The insertion device of claim 1, wherein the bearing region is arranged in a balloon catheter.

10. The insertion device of claim 1, wherein the balloon and/or the membrane is/are formed from nylon or polyamide.

11. The insertion device of claim 1, wherein the balloon and/or the membrane is/are formed from a material selected from the group consisting of ethylene vinyl acetate, polyvinyl chloride, olefin copolymers, olefin homopolymers, polyethylene, polyethylene types, polyurethane, polyethylene terephthalate, acrylonitrile polymers, acrylonitrile co-polymers, acrylonitrile mixtures, resins, and ionomer resins.

12. The insertion device of claim 1, wherein, when pressure is applied to the resilient membrane, the guide wire, balloon catheter and guide catheter can be fixed relative to one another in their relative orientation.
